(19) United States
(12) Patent Application Publication
(10) Pub. No.: US 2025/0011205 A1
(43) Pub. Date: Jan. 9, 2025

(54) METHOD FOR ACCURATELY CONTROLLING DISSOLVED OXYGEN OF AEROBIC WASTEWATER TREATMENT SYSTEM

(71) Applicant: NANJING UNIVERSITY, Nanjing (CN)

(72) Inventors: Lin YE, Nanjing (CN); Yanyan Ma, Nanjing (CN)

(21) Appl. No.: 18/790,713

(22) Filed: Jul. 31, 2024

(30) Foreign Application Priority Data
Apr. 16, 2024 (CN) 202410454596.6

(51) Int. Cl. C02F 3/02

(52) U.S. Cl. CPC C02F 3/02; C02F 2209/003; C02F 2209/006; C02F 2209/08; C02F 2209/22; C02F 2209/38

(57) ABSTRACT

The present invention belongs to the technical field of wastewater treatment and more specifically relates to a method for accurately controlling dissolved oxygen of an aerobic wastewater treatment system. The system integrates the functions of online monitoring and dissolved oxygen control of system parameters of a wastewater treatment plant, can acquire data of the dissolved oxygen concentration, openings of air valves, air quantity of a fan, influent quality and quantity of the wastewater treatment system in real time, automatically calculates ideal values of the air quantity of the fan and the openings of the air valves according to a target value of the dissolved oxygen concentration by a server, screens an optimal adjusting mode on the basis of a certain condition, and finally sends an instruction to the fan or the air valves to realize the accurate control of dissolved oxygen of the wastewater treatment system.

### TECHNICAL FIELD

The present invention belongs to the technical field of wastewater treatment and more specifically relates to a method for accurately controlling dissolved oxygen of an aerobic wastewater treatment system.

### BACKGROUND

Wastewater treatment plants play a vital role in today's society. However, the conventional wastewater treatment method often faces the challenges of high energy consumption, high cost, complex operation and the like. In response to these challenges, the application of a machine learning technology is bringing a whole new solution to the wastewater treatment plants, and the application of machine learning models in the wastewater treatment plants has become an important means for improving operation efficiency, reducing cost, and optimizing processes.

In the prior art, in the wastewater treatment plants, machine learning method is widely applied to operation monitoring: by collecting a large amount of data acquired by equipment such as a sensor, a flowmeter, a water quality testing instrument and the like, the machine learning model can perform real-time supervision and analysis to the wastewater treatment process. These models can recognize the abnormal conditions and make early warning in advance to help the operation personnel to respond fast, thereby reducing potential loss and risk.

The energy consumption of the wastewater treatment plants has always been the focus of attention in the industry, wherein the power consumed by an aeration process of an aerobic wastewater treatment occupies a considerable proportion. In addition, a conventional aeration system needs to be continuously manually adjusted, which not only wastes time and labor, but also has the risk of operation errors. If a machine learning model can be introduced to replace manual work for adjusting the aeration process, a plurality of advantages are brought.

In addition, wastewater treatment plants often adopt a mode of parallel operation of a plurality of fans, and the air quantity generated by the fans can be gathered into a pipeline and is controlled by air valves to be distributed to each biochemical tank. When the deviation of the dissolved oxygen concentration and the set value is not large, the air valve opening of each biochemical tank is adjusted to control the dissolved oxygen concentration. However, since the air quantity of the fan is constant, the change of a single air valve opening influences the quantity of air introduced into other air valves, namely the dissolved oxygen concentration of other biochemical tanks is also influenced. Moreover, when the deviation of the dissolved oxygen and the set value is too large, the air quantity of the fan can be adjusted. Therefore, the control on the air quantity of the fan and the air valves opening is very important and complex. The adjustment by the experience of a technician is inevitably insufficient in accuracy, large in error and the like.

Chinese patent document CN201620880870.7 discloses a dissolved oxygen monitoring system for wastewater treatment. The monitoring system can automatically drive a buzzer and a warning light to complete the on-site alarm work through the cooperation of a feedback module and a microprocessor, such that the operational personnel on the site can take relevant oxygenation measures to remedy in time. However, the patent does not provide a technical solution to the problem that the dissolved oxygen concentration needs to be manually controlled and does not provide any technical suggestion.

### SUMMARY

### 1. Problem to be Solved

Aiming at the problem that the dissolved oxygen concentration of biochemical tanks in an aerobic wastewater treatment system needs to be manually controlled in the prior art, the present invention provides a method for accurately controlling dissolved oxygen of an aerobic wastewater treatment system by using a deep learning model to intelligently control the dissolved oxygen concentration.

### 2. Technical Solution

The present invention provides a method for accurately controlling dissolved oxygen of an aerobic wastewater treatment system, comprising the following steps:
S1, collecting data of influent quality, influent quantity, dissolved oxygen concentrations of a first biochemical tank and a second biochemical tank, a first air valve opening and a second air valve opening, and air quantity of a fan of an aerobic wastewater treatment system using a data acquisition and control module, and transmitting the data to a server;
S2, constructing a machine learning matrix on the basis of the data of the influent quality, the influent quantity, the dissolved oxygen concentrations of a first biochemical tank and a second biochemical tank, the first air valve opening and the second air valve opening, and the air quantity of the fan of the aerobic wastewater treatment system;
S3, establishing a deep learning model among the data of the influent quality, the influent quantity, the dissolved oxygen concentrations of a first biochemical tank and a second biochemical tank, the first air valve opening and the second air valve opening, and the air quantity of the fan of the aerobic wastewater treatment system; and training the deep learning model on the basis of the data matrix constructed in step S2;
S4, setting target values of the dissolved oxygen concentrations of a first biochemical tank and a second biochemical tank in server; and
S5, when the dissolved oxygen concentrations of a first biochemical tank and a second biochemical tank deviate from the target values, calculating suggested adjustment values of the first air valve opening and the second air valve opening, and the air quantity of the fan on the basis of the model constructed in step S3, and maintaining the dissolved oxygen concentrations of a first biochemical tank and a second biochemical tank at the target values by adjusting the first air valve opening and the second air valve opening, and the air quantity of the fan.

Further, in step S1, the data of the influent quality comprises data of chemical oxygen demand concentration and ammonia nitrogen concentration of influent.

Further, in step S1, an acquisition time interval of the data of the influent quality, the influent quantity, the dissolved oxygen concentrations of a first biochemical tank and a second biochemical tank, the first air valve opening and the second air valve opening, and the air quantity of the fan of the aerobic wastewater treatment system is a fixed value,
wherein, the acquisition time interval of the data of the influent quality and the influent quantity is less than or equal to 60 minutes, and the acquisition time interval of the data of the dissolved oxygen concentrations of a first biochemical tank and a second biochemical tank, the first air valve opening and the second air valve opening, and the air quantity of the fan is 20-60 seconds.

Further, in step S2, constructing the machine learning matrix is as follows:

$$[ \begin{matrix}{t}_{1} & OP {1}_{1} & OP {2}_{1} & {FL}_{1} & {COD}_{1} & {N}_{1} & int {FL}_{1} & DO {1}_{1} & DO {2}_{1} \\ {t}_{2} & OP {1}_{2} & OP {2}_{2} & {FL}_{2} & {COD}_{2} & {N}_{2} & int {FL}_{2} & DO {1}_{2} & DO {2}_{2} \\ ⋮ & ⋮ & ⋮ & ⋮ & ⋮ & ⋮ & ⋮ & ⋮ & ⋮ \\ {t}_{m} & OP {1}_{m} & OP {2}_{m} & {FL}_{m} & {COD}_{m} & {N}_{m} & int {FL}_{m} & DO {1}_{m} & DO {2}_{m}\end{matrix} ]$$

t₁-tₘ are the time of each record, OP1₁-OP1ₘ are the first air valve opening corresponding to each record, OP2₁-OP2ₘ are the second air valve opening corresponding to each record, FL₁-FLₘ are the air quantities of the fan corresponding to each record, COD₁-CODₘ are chemical oxygen demands of influent corresponding to each record, N₁-Nₘ are ammonia nitrogen concentrations of the influent corresponding to each record, intFL₁-intFLₘ are influent quantities corresponding to each record, DO1₁-DO1ₘ are the dissolved oxygen concentrations of a first biochemical tank corresponding to each record, and DO2₁-DO2ₘ are the dissolved oxygen concentrations of a second biochemical tank corresponding to each record.

Further, the deep learning model in step S3 is a long short-term memory model constructed on the basis of a long short-term memory LSTM network.

Further, the long short-term memory model requires more than or equal to 6 days of data to train before use.

Further, in a use process of the long short-term memory model, training and learning are performed again every 30 minutes according to the updated data.

By using the technical solution, the model can be continuously adapted to the current aerobic wastewater treatment system by training and learning again according to the updated data every 30 minutes, and the dissolved oxygen concentrations of a first biochemical tank and a second biochemical tank can be more accurately adjusted.

Further, in step S4, a target value range of the dissolved oxygen concentrations of a first biochemical tank and a second biochemical tank is 1-5 mg/L; and when a difference value of the measured dissolved oxygen concentration value and the target value is greater than 0.2 mg/L, the dissolved oxygen concentrations of a first biochemical tank and a second biochemical tank are considered to deviate from the target value.

Further, in step S4, a method for setting the target value of the dissolved oxygen concentrations of a first biochemical tank and a second biochemical tank in the server is to input the set target value into the server by using a user terminal.

By using the technical solution, the server gives the fan and valves adjustment suggestions according to the current influent quality, influent quantity and dissolved oxygen concentrations of the aerobic wastewater treatment system. An optimal adjusting instruction obtained by screening is transmitted to the fan and/or the valves through the data acquisition and control module, such that the dissolved oxygen concentrations in the biochemical tanks can meet the requirements.

Further, in step S5, a specific method for calculating adjustment values of the first air valve opening and the second air valve opening, and the air quantity of the fan is as follows: when a dissolved oxygen concentration detected by a first online dissolved oxygen detector or a second online dissolved oxygen detector deviates from a target value, inputting data of influent quality, influent quantity, dissolved oxygen concentrations of a first biochemical tank and a second biochemical tank, the first air valve opening and the second air valve opening, and air quantity of the fan of an aerobic wastewater treatment system at the time of 0-T, and target values of the dissolved oxygen concentrations of a first biochemical tank and a second biochemical tank at the time of T+1 into a long short-term memory model for prediction, and respectively calculating to obtain numerical values required to be adjusted of the first air valve opening and the second air valve opening or the air quantity of the fan at the time of T+1. T and T+1 are separated by a time step, and acquisition time intervals of multi-data monitoring equipment are different. When the time step of an input data matrix is less than the parameter acquisition interval, values in the single acquisition interval of the parameter are repeatedly applied to the multiple time steps; and when the time step is greater than the parameter acquisition interval, the average value within the multiple acquisition intervals of the parameter is applied to the time step.

Further, in step S5, when the air quantity of the fan needs to be adjusted to be greater, if the suggested adjustment values of the first air valve opening and/or the second air valve opening given by server are within adjustable ranges, the first air valve opening or the second air valve opening is preferably adjusted; and if the suggested adjustment values of the first air valve opening and the second air valve opening given by server exceed the adjustable ranges, and the suggested adjustment value of the air quantity of the fan given by server is within an adjustable range, the air quantity of the fan is preferably adjusted. Compared with a solution of directly increases the air quantity of the fan, the solution is more energy-saving and consumption-reducing.

Further, in step S5, when the air quantity of the fan needs to be adjusted to be lower, if the suggested adjustment value of the air quantity of the fan given by server is within the adjustable range, the air quantity of the fan is preferably adjusted; and if the suggested adjustment value of the air quantity of the fan given by server exceeds the adjustable range, and the suggested adjustment values of the first air valve opening and/or the second air valve opening given by server are within the adjustable ranges, the first air valve opening or the second air valve opening is preferably adjusted. Compared with a solution of preferably adjusting the first air valve opening or the second air valve opening, the solution can reduce aeration quantities of the wastewater treatment plant, thereby achieving an energy-saving effect.

Further, when the air quantity of the fan needs to be adjusted to be greater, if the suggested adjustment values of the first air valve opening and the second air valve opening given by server are both within the adjustable ranges, the air valve that is suggested for a larger adjustment is adjusted.

Further, when the air quantity of the fan needs to be adjusted to be greater, if the suggested adjustment values of the first air valve opening and the second air valve opening given by the server are both within the adjustable ranges, the first air valve opening is suggested to be adjusted to be greater by the server, and the second air valve opening is suggested to be adjusted to be lower, then the first air valve opening is adjusted.

Further, when the air quantity of the fan needs to be adjusted to be greater, if the suggested adjustment values of the first air valve opening and the second air valve opening given by the server are both within the adjustable ranges, the first air valve opening is suggested to be adjusted to be lower by the server, and the second air valve opening is suggested to be adjusted to be greater, then the second air valve opening is adjusted.

Further, when the air quantity of the fan needs to be adjusted to be lower, if the suggested adjustment value of the air quantity of the fan given by server exceeds the adjustable range, and the suggested adjustment values of the first air valve opening and the second air valve opening given by server are both within the adjustable ranges, the air valve that is suggested for a smaller adjustment is adjusted.

Further, when the air quantity of the fan needs to be adjusted to be lower, if the suggested adjustment value of the air quantity of the fan given by the server exceeds the adjustable range, the suggested adjustment values of the first air valve opening and the second air valve opening given by the server are both within the adjustable ranges, the first air valve opening is suggested to be adjusted to be greater by the server, and the second air valve opening is suggested to be adjusted to be lower, then the second air valve opening is adjusted.

Further, when the air quantity of the fan needs to be adjusted to be lower, if the suggested adjustment value of the air quantity of the fan given by the server exceeds the adjustable range, the suggested adjustment values of the first air valve opening and the second air valve opening given by the server are both within the adjustable ranges, the first air valve opening is suggested to be adjusted to be lower by the server, and the second air valve opening is suggested to be adjusted to be greater, then the first air valve opening is adjusted.

Further, in step S5, when the air quantity of the fan, the first air valve opening and the second air valve opening are both suggested to be adjusted to be greater or lower by server, a multi-parameter adjustment strategy is implemented, the three parameters are simultaneously identified whether the adjustable ranges are exceeded or not, and the parameters which are not exceeded are simultaneously adjusted. When the air quantity of the fan, the first air valve opening and the second air valve opening given by the server are both suggested to be adjusted to be greater or lower, the dissolved oxygen of a first biochemical tank and a second biochemical tank reaches a limit value simultaneously, and multiple parameters are simultaneously adjusted. Compared with a solution of only adjusting a single parameter, the solution responds more quickly and prevents the occurrence of emergency.

Further, in step S5, when the air quantity of the fan given by the server is suggested to be adjusted to be greater, it is impossible to suggest the first air valve opening and the second air valve opening to be both adjusted to be lower; and when the air quantity of the fan given by the server is suggested to be adjusted to be lower, it is impossible to suggest the first air valve opening and the second air valve opening to be both adjusted to be greater.

Further, in step S5, when the air quantity of the fan, the first air valve opening and the second air valve opening given by server all exceed adjustable ranges, manual checking and confirmation are performed.

It should be noted that when the air quantity of the fan is increased, both aeration quantities of a first biochemical tank and a second biochemical tank are increased, the dissolved oxygen concentrations are increased, and when the air quantity of the fan was decreased in the same way.

Increasing the first air valve opening and keeping the second air valve unchanged, the aeration amount of a first biochemical tank is increased, and the dissolved oxygen concentration is increased. But since the air quantity of the fan is unchanged, the aeration quantity and the dissolved oxygen concentration is reduced. Increasing the second air valve opening and keeping the first air valve unchanged in the same way. That is, when the air quantity of the fan is fixed, the total ventilation quantity of the first air valve and the second air valve is fixed, and the total aeration quantity of a first biochemical tank and a second biochemical tank is fixed.

Further, adjustable ranges of the first air valve opening and the second air valve opening are 30-100%.

Further, an adjustable range of the air quantity of the fan is 20-90% of a maximum air quantity.

Further, the aerobic wastewater treatment system on the basis of the method for accurately controlling dissolved oxygen of an aerobic wastewater treatment system comprises:
a first biochemical tank and a second biochemical tank used for performing an aerobic wastewater treatment;
a fan used for controlling the total aeration quantity of a first biochemical tank and a second biochemical tank;
a first air valve positioned between the fan and the first biochemical tank, and communicating to the fan and the first biochemical tank;
a second air valve positioned between the fan and the second biochemical tank, and communicating to the fan and the second biochemical tank;
a first online dissolved oxygen detector used for detecting a dissolved oxygen concentration in the first biochemical tank; and
a second online dissolved oxygen detector used for detecting a dissolved oxygen concentration in the second biochemical tank; and
an online influent quality and quantity data detector comprising an online COD analyzer, an online ammonia nitrogen analyzer and a flowmeter, and used for detecting the influent quality and quantity of the wastewater treatment system.

Further, the aerobic wastewater treatment system further comprises: a data acquisition and control module connected to the fan, the first air valve, the second air valve, the first online dissolved oxygen detector, the second online dissolved oxygen detector and the online influent quality and quantity data detector.

The data acquisition and control module is used for acquiring the data of the air quantity of the fan, the first air valve opening and the second air valve opening, the dissolved oxygen concentrations of a first biochemical tank and a second biochemical tank, the influent quality, and the influent quantity, and controlling the air quantity of the fan, the first air valve opening and the second air valve opening.

Further, the aerobic wastewater treatment system further comprises: a server connected to the data acquisition and control module.

After the server acquires and stores the data of the air quantity of the fan, the first air valve opening and the second air valve opening, the dissolved oxygen concentrations of a first biochemical tank and a second biochemical tank, the influent quality, and the influent quantity, suggested adjustment values of the air quantity of the fan, the first air valve opening and the second air valve opening are calculated, an optimal adjusting mode is screened, and then an adjustment instruction is transmitted to the data acquisition and control module.

Further, the aerobic wastewater treatment system further comprises a user terminal connected to the server.

Target values of dissolved oxygen concentrations of a first biochemical tank and a second biochemical tank are input into the user terminal by a user; and the user terminal transmits the target values to the server.

Further, the connection mode between the data acquisition and control module and the fan, the first air valve, the second air valve, the first online dissolved oxygen detector, the second online dissolved oxygen detector and the online influent quality and quantity data detector, between the server and the data acquisition and control module, and between the user terminal and the server can be wired or wireless.

By using the technical solution, the data acquisition and control module acquires the data of the influent quality, the influent quantity, the first air valve opening and the second air valve opening, and the air quantity of the fan in real time; then the data is transmitted to a server in real time for storage and analysis; a dynamic simulation model of the aerobic wastewater treatment system is constructed on the basis of big data analysis and a machine learning method, and the server respectively gives suggested adjustment values of the air quantity of the fan, the first air valve opening and the second air valve opening; and the server selects an optimal adjusting mode according to an adjusting priority, sends an optimal adjusting instruction to the data acquisition and control module, controls the air quantity of the fan, the first air valve opening and the second air valve opening, and accurately controls dissolved oxygen of the biochemical tanks.

In the prior art, the dissolved oxygen concentration control of a wastewater treatment plant is usually based on manual regulation and control, and has high requirement on the experience of workers, high labor cost, and high energy consumption. The present invention uses the long short-term memory model to calculate and screen adjusting modes of reasonable valves opening and air quantity of a fan according to an artificially preset dissolved oxygen concentration target value, and automatically sends instructions to the fan and the air valves, thereby automatically and accurately controlling dissolved oxygen. The present invention can save the labor cost of the wastewater treatment plant, also maintain the dissolved oxygen concentrations of the biochemical tanks in a stable range, improve process stability, reduce accidents such as effluent deterioration caused by out-of-control dissolved oxygen in the wastewater treatment plant, and also reduce the total aeration quantity and reduce energy consumption. The present invention can realize intelligent control by using the long short-term memory model to relieve the problems.

### 3. Beneficial Effects

Compared with the prior art, the present invention has the following beneficial effects: Through machine learning, the present invention controls the dissolved oxygen of the wastewater treatment system in a reasonable range, thereby reducing waste of electric power of the aeration fan, also greatly reducing the labor cost, and ensuring the high-efficiency and low-consumption operation of a biological treatment process in the wastewater treatment system.

More specifically, the present invention has the following beneficial effects:

(1) The machine learning model used by the present invention can accurately monitor the water quality and aeration requirements and make quick and accurate adjustment according to real-time data, thereby improving the treatment efficiency and reducing the energy waste.

The machine can work continuously and stably without influence by factors such as personnel fatigue and emotion, ensures the operation stability and reliability of an aeration system, has a full-automatic regulation and control mode without labor cost, reduces the influence of human errors on the treatment effect, and further improves the overall quality and efficiency of wastewater treatment.

(2) The present invention intelligently regulates and controls the dissolved oxygen by using a long short-term memory (LSTM) model, can realize accurate, self-adaptive and intelligent control, and brings whole new possibility for the operation management of a wastewater treatment plant.

Compared with a traditional method, the used LSTM model can realize accurate control, adapt to different operating conditions, and realize generalization prediction of unknown conditions and automatic control decision making.

(3) In the present invention, the used LSTM model and an optimization algorithm are used to realize an optimal control strategy to further improve the processing efficiency, save energy and reduce consumption. When the air quantity of the fan needs to be adjusted to be greater, a first air valve opening and a second air valve opening are preferably adjusted; when the air quantity of the fan needs to be adjusted to be lower, the air quantity of the fan is preferably adjusted to be lower and the energy consumption of the fan can be reduced; and when the air quantity of the fan, the first air valve opening and the second air valve opening are all required to be adjusted to be greater or lower, the multiple parameters are adjusted simultaneously, the response is quicker, and the occurrence of emergency is prevented.

(4) The present invention does not need to perform large-scale structure reconstruction on the wastewater treatment plant and has low construction cost.

### BRIEF DESCRIPTION OF THE DRAWINGS

FIG. 1 is a schematic diagram showing a control flow of the method for accurately controlling dissolved oxygen of an aerobic wastewater treatment system in example 1;

FIG. 2 is a schematic diagram showing control solutions and adjusting priority of the first air valve, the second air valve and the fan in example 1;

FIG. 3 is a schematic diagram showing specific preferred adjusting modes of the first air valve, the second air valve and the fan in example 1;

FIG. 4 is a diagram showing the implementation effect of the method for accurately controlling dissolved oxygen of an aerobic wastewater treatment system in example 1; and

FIG. 5 is a diagram showing the implementation effect of the method for manually controlling dissolved oxygen of an aerobic wastewater treatment system in comparative example 1.

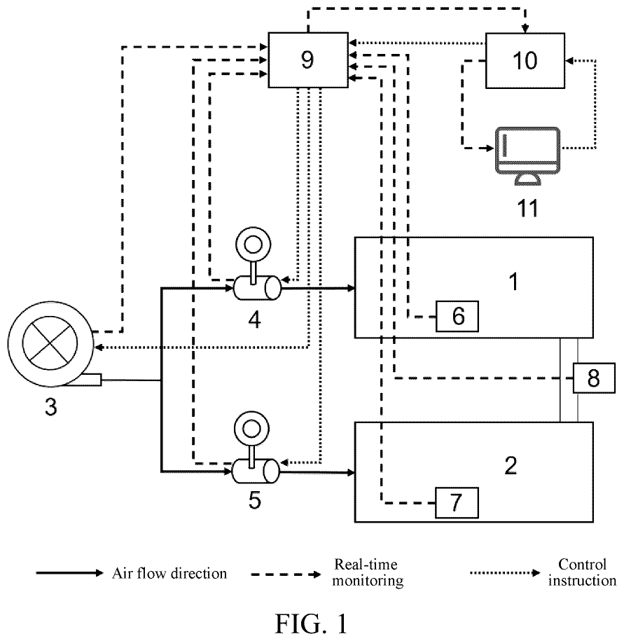

In the figures,
1. a first biochemical tank; 2. a second biochemical tank; 3. a fan; 4. a first air valve; 5. a second air valve; 6. a first online dissolved oxygen detector; 7. a second online dissolved oxygen detector; 8. an online influent quality and quantity data detector; 9. a data acquisition and control module; 10. a server; and 11. a user terminal; and
“FL” indicates air quantity of the fan; “OP1” indicates the first air valve opening; “OP2” indicates the second air valve opening; “DO1” indicates the dissolved oxygen concentration of the first biochemical tank; “DO2” indicates the dissolved oxygen concentration of the second biochemical tank; “T” indicates that suggestion given by a server is to be adjusted to be greater; and “i” indicates suggestion given by a server is to be adjusted to be lower.

### DETAILED DESCRIPTION

It should be noted that when an element is referred to be “mounted” to another element, it can be directly on the other element or the two elements may be directly integrated; and when an element is referred to be “connected” to another element, it can be directly connected to the other element or the two elements may be directly integrated. In addition, the terms such as “upper”, “lower”, “left”, “right” and “middle” referred in the present description are also only used for clarity of description, but not used to limit the implementable scope. The changes or adjustments of the relative relationship thereof can be considered as the implementable scope of the present invention without substantial technical changes.

Unless defined otherwise, all the technical and scientific terms used herein have the same meaning as commonly understood by one of ordinary skill in the art to which the present invention belongs; and the term “and/or” used herein includes any and all combinations of one or more related listed items.

If no specific conditions are specified in the examples, the examples will be conducted according to conventional conditions or the conditions recommended by the manufacturer. All of the used reagents or instruments which are not specified with manufacturers are conventional commercially-available products.

The term “about” used herein is used to provide the flexibility and inaccuracy associated with the given terms, measures or values. The degree of flexibility for a particular variable may be readily determined by one skilled in the art.

The term “adjacent” used herein refers to two structures or elements being in proximity. Specifically, elements identified as “adjacent” may abut or be connected. Such elements may also be near or proximate to each other without necessarily contacting each other. In some cases, the precise degree of proximity may depend on the particular context.

The term “at least one in . . . ” used herein is intended to be synonymous with the term “one or more in . . . ”. For example, “at least one in A, B and C” explicitly includes A alone, B alone, C alone and a respective combination thereof.

Data of concentrations, amounts and other numerical values may be presented herein in a range format. It should be understood that such range format is used merely for convenience and brevity, and should be interpreted flexibly to include not only the numerical values explicitly recited as a range limit, but also to include all the individual numerical values or sub-ranges encompassed within that range as if each numerical value and sub-range is explicitly recited. For example, a numerical value range of about 1 to about 4.5 should be interpreted to include not only the explicitly recited limit values of 1 to about 4.5, but also include individual numbers (such as 2, 3 and 4) and sub-ranges (such as 1 to 3, 2 to 4, etc.). The same principle applies to a range reciting only one numerical value, such as “less than about 4.5”, which should be interpreted as including all such values and ranges. Further, such interpretation should apply regardless of the breadth of the described ranges or characteristics.

Any steps recited in any method or process claim (e.g., steps S1, S2, S3 . . . or steps (1), (2), (3) . . . or steps 1), 2), 3) . . . ) may be performed in any order and are not limited to the order presented in the claims.

A limitation of method+function or step+function is only employed if all the following conditions exist within the limitation of a particular claim: a) “a method for . . . ” or “a step for . . . ” is explicitly recited; and b) the corresponding functions are explicitly recited. The structure, material, or acts of support method+function are explicitly recited in the description herein. Therefore, the scope of the present invention is determined only by the appended claims and legal equivalents thereof, but not by the description and examples given herein. The present invention is further described below with reference to specific examples.

### Example 1

The present example provided a method for accurately controlling dissolved oxygen of an aerobic wastewater treatment system specifically as follows:

In the present example, the aerobic wastewater treatment system used an activated sludge process to treat wastewater of an industrial park, a chemical oxygen demand range of influent was 100-500 mg/L, the aerobic wastewater treatment system comprised a first biochemical tank 1 and a second biochemical tank 2, a group of variable frequency fans 3 were used for daily oxygen supply of the first biochemical tank 1 and the second biochemical tank 2, and a first air valve 4 and a second air valve 5 were respectively arranged before air led to the two biochemical tanks. An online influent quality and quantity data detector 8 was used for monitoring influent quantity, chemical oxygen demand and ammonia nitrogen concentration of the aerobic wastewater treatment system in real time, wherein an acquisition interval of data of the influent quantity was 30 minutes, and an acquisition interval of data of the chemical oxygen demand and the ammonia nitrogen concentration was 1 hour. A first online dissolved oxygen detector 6 and a second online dissolved oxygen detector 7 were respectively arranged in the first biochemical tank 1 and the second biochemical tank 2, and an acquisition interval of data in real time was 1 minute. An acquisition interval of data of air quantity of the fan 3 and openings of the two air valves was also 1 minute. The monitored various data were transmitted to a central control system (a user terminal 11) in real time so as to facilitate observation and operation of workers.

In the present example, the online influent quality and quantity data detector 8 comprised an online COD analyzer, an online ammonia nitrogen analyzer and a flowmeter.

The first online dissolved oxygen detector 6, the second online dissolved oxygen detector 7, the online COD analyzer, the online ammonia nitrogen water quality analyzer, and the flowmeter in the present example were all conventional products that can be purchased commercially, and the products and models were not limited.

The method for accurately controlling dissolved oxygen of an aerobic wastewater treatment system was applied to the aerobic wastewater treatment system, monitored data of the influent quality and quantity, data of dissolved oxygen of the first biochemical tank 1 and the second biochemical tank 2, and data of the air quantity of the fan 3 and the openings of the two air valves, and transmitted the data to a server in real time. According to the current parameter conditions and a set target value of the dissolved oxygen concentration, the server respectively gave suggested values of the air valves openings of the first biochemical tank 1 and the second biochemical tank 2 and the air quantity of the fan 3.

In the present example, a user input the set target value of the dissolved oxygen to the server 10 by using the user terminal 11.

In this example, a Keras framework was used to establish a long short-term memory model.

Specifically, the data of the influent quality, the influent quantity, the dissolved oxygen concentrations DO1 and DO2 of a first biochemical tank 1 and a second biochemical tank 2, the openings OP1 and OP2 of the first air valve 4 and the second air valve 5, and the air quantity FL of a fan 3 at the time of 0-T, and target values of the dissolved oxygen concentrations DO1(T+1) and DO2(T+1) of a first biochemical tank 1 and a second biochemical tank 2 at the time of T+1 were input into a long short-term memory model (LSTM) for prediction, and numerical values required to be adjusted of the opening OP1(T+1) of the first air valve 4 and the opening OP2(T+1) of the second air valve 5 or the air quantity FL(T+1) of the fan 3 at the time of T+1 were respectively obtained by calculating.

On the basis the principle of energy saving, when the air quantity of the fan 3 needed to be adjusted to be greater, the air valves openings were preferably adjusted, and when the air valves openings were both suggested to exceed an adjustable range, the air quantity of the fan 3 was preferably adjusted; when the air quantity of the fan 3 needed to be adjusted to be lower, the air quantity of the fan 3 was preferably adjusted, and when the air quantity of the fan 3 was suggested to exceed an adjustable range, the air valves openings were preferably adjusted.

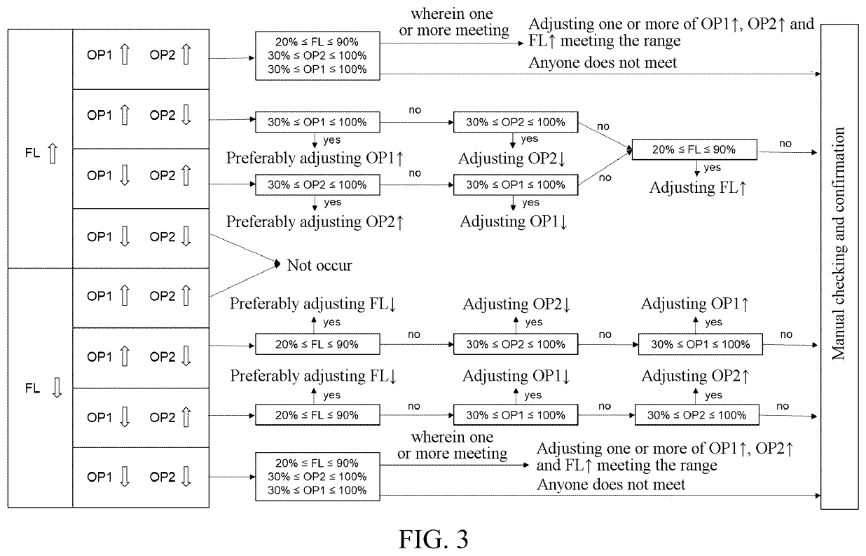

As shown in FIG. 3, when the air quantity of the fan 3 needs to be adjusted to be greater, if the suggested adjustment values of the openings of the first air valve 4 and the second air valve 5 given by the server 10 are both within the adjustable ranges, the air valve that is suggested for a larger adjustment is adjusted. Specifically,

(1) when the air quantity of the fan 3 needs to be adjusted to be greater, if the suggested adjustment values of the openings of the first air valve 4 and the second air valve 5 given by the server 10 are both within the adjustable ranges, the opening of the first air valve 4 is suggested to be adjusted to be greater, and the opening of the second air valve 5 is suggested to be adjusted to be lower, then the opening of the first air valve 4 is preferably adjusted.

(2) When the air quantity of the fan 3 needs to be adjusted to be greater, if the suggested adjustment values of the openings of the first air valve 4 and the second air valve 5 given by the server 10 are both within the adjustable ranges, the opening of the first air valve 4 is suggested to be adjusted to be lower, and the opening of the second air valve 5 is suggested to be adjusted to be greater, then the opening of the second air valve 5 is preferably adjusted.

As shown in FIG. 3, when the air quantity of the fan 3 needs to be adjusted to be lower, if the suggested adjustment value of the air quantity of the fan 3 given by the server 10 exceeds the adjustable range, and the suggested adjustment values of the openings of the first air valve 4 and the second air valve 5 given by the server 10 are both within the adjustable ranges, the air valve that is suggested for a smaller adjustment is adjusted. Specifically,

(1) when the air quantity of the fan 3 needs to be adjusted to be lower, if the suggested adjustment value of the air quantity of the fan 3 given by the server 10 exceeds the adjustable range, the suggested adjustment values of the openings of the first air valve 4 and the second air valve 5 given by the server 10 are both within the adjustable ranges, the opening of the first air valve 4 is suggested to be adjusted to be greater, and the opening of the second air valve 5 is suggested to be adjusted to be lower, then the opening of the second air valve 5 is preferably adjusted.

(2) When the air quantity of the fan 3 needs to be adjusted to be lower, if the suggested adjustment value of the air quantity of the fan 3 given by the server 10 exceeds the adjustable range, the suggested adjustment values of the openings of the first air valve 4 and the second air valve 5 given by the server 10 are both within the adjustable ranges, the opening of the first air valve 4 is suggested to be adjusted to be lower, and the opening of the second air valve 5 is suggested to be adjusted to be greater, then the opening of the first air valve 4 is preferably adjusted.

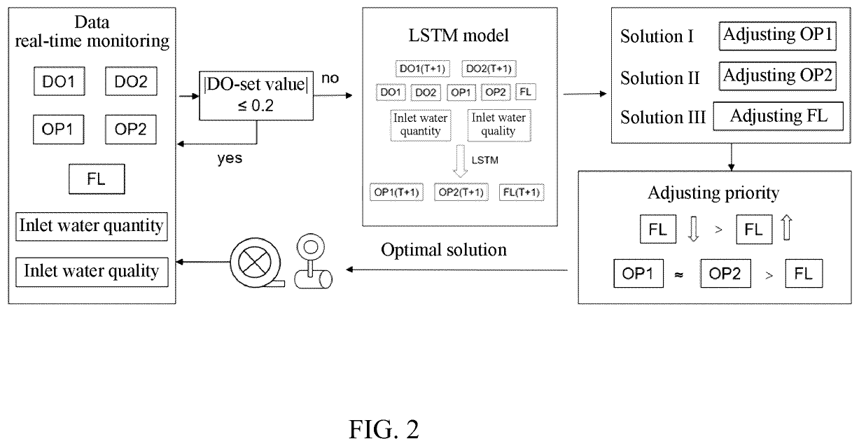

When the air quantity of the fan 3 and the openings of the first air valve 4 and the second air valve 5 given by the server were both suggested to be adjusted to be greater or lower, a multi-parameter adjustment strategy was implemented, the three parameters were simultaneously identified whether the adjustable ranges were exceeded or not, and the parameters which were not exceeded were simultaneously adjusted; when the air quantity of the fan 3 given by the server was suggested to be adjusted to be greater, it was impossible to suggest the openings of the first air valve 4 and the second air valve 5 to be both adjusted to be lower; when the air quantity of the fan 3 given by the server was suggested to be adjusted to be lower, it was impossible to suggest the openings of the first air valve 4 and the second air valve 5 to be both adjusted to be greater; and when the air quantity of the fan 3, and the suggested adjustment values of the openings of the first air valve 4 and the second air valve 5 given by the server all exceed adjustable ranges, an alarm was sent to the central control system, and manual checking and confirmation were performed. The specific adjusting modes and priority of the fan 3 and the air valves were shown in FIGS. 2 and 3.

An optimal adjusting mode was finally screened out according to the adjusting priority and an instruction was sent to the electronic air valves or the fan 3 so as to accurately control the dissolved oxygen concentrations.

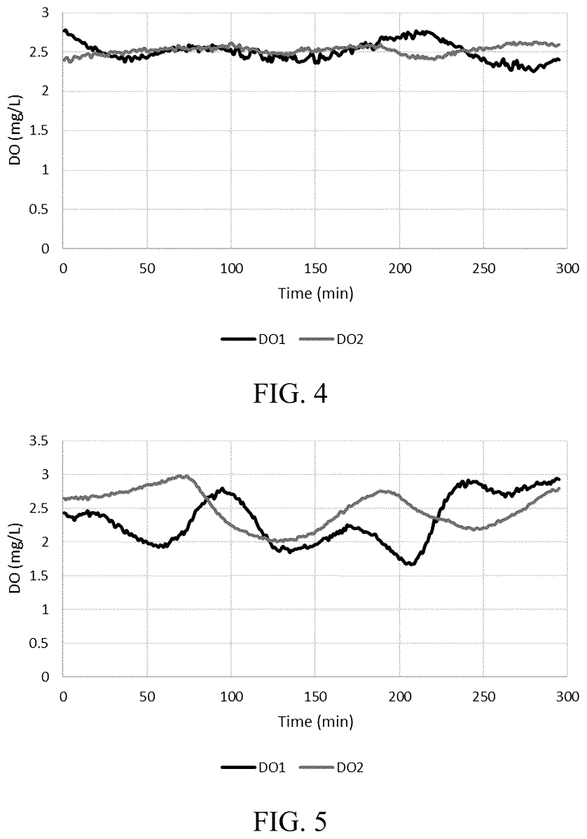

Before the method was formally used for automatic control, 6 days of manual adjustment data were used for training and learning the long short-term memory model, a time step of a machine learning matrix was 1 minute, the ratio of sizes of a training set to a testing set was 9:1, the model timestep was 10, the epoch of model training was 50, and the batch size was 100. The target values of the dissolved oxygen concentrations DO1 and DO2 of the first biochemical tank 1 and the second biochemical tank 2 were set to 2.5 mg/L, automated control was performed by using the model, and changes in the dissolved oxygen concentrations within 5 hours were tested. The implementation effect of the method was as shown in FIG. 4.

In the present example, the following specific adjusting processes were provided as a specific application of the method for accurately controlling dissolved oxygen of an aerobic wastewater treatment system of the present example.

First adjusting process: the target dissolved oxygen concentrations of the first biochemical tank 1 and the second biochemical tank 2 were 2.5 mg/L, DO1 was in an ascending trend (2.6 mg/L), DO2 was in a descending trend (2.3 mg/L), and DO2 reached a target value range threshold of 2.5-0.2 mg/L. Adjusting modes of the three parameters were respectively obtained by model calculation: (1) increasing the air quantity FL of the fan 3 from 53% to 65%; (2) decreasing the opening OP1 of the first air valve 4 from 51% to 45%; and (3) increasing the opening OP2 of the second air valve 5 from 59% to 72%. The obtained optimal adjusting mode was that the opening OP2 of the second air valve 5 was adjusted from 59% to 72%, such that the dissolved oxygen concentration DO2 of the second biochemical tank 2 was increased and the dissolved oxygen concentration DO1 of the first biochemical tank 1 was decreased. DO1 and DO2 were maintained in a range of 2.3-2.7 mg/L after the adjustment.

Second adjusting process: the target dissolved oxygen concentrations of the first biochemical tank 1 and the second biochemical tank 2 were 2.5 mg/L, DO1 was in an ascending trend (2.7 mg/L), DO2 was in an ascending trend (2.4 mg/L), and DO1 reached a target value range threshold of 2.5+0.2 mg/L. Adjusting modes of the three parameters were respectively obtained by model calculation: (1) decreasing the air quantity FL of the fan 3 from 34% to 18%; (2) decreasing the opening OP1 of the first air valve 4 from 52% to 37%; and (3) increasing the opening OP2 of the second air valve 5 from 69% to 84%. The air quantity of the fan 3 was adjusted in first. However, since the adjusting mode of the air quantity FL of the fan 3 <20%, it exceeded a set normal adjusting range. Therefore, the obtained optimal adjusting mode was that the opening OP1 of the first air valve 4 was adjusted from 52% to 37%, such that the dissolved oxygen concentration DO1 of the first biochemical tank 1 was decreased and the dissolved oxygen concentration DO2 of the second biochemical tank 2 was increased. DO1 and DO2 were maintained in a range of 2.3-2.7 mg/L after the adjustment.

Third adjusting process: the target dissolved oxygen concentrations of the first biochemical tank 1 and the second biochemical tank 2 were 2.5 mg/L, DO1 was in a descending trend (2.3 mg/L), DO2 was in a descending trend (2.3 mg/L), and DO1 and DO2 reached a target value range threshold of 2.5-0.2 mg/L. Adjusting modes of the three parameters were respectively obtained by model calculation: (1) increasing the air quantity FL of the fan 3 from 43% to 58%; (2) increasing the opening OP1 of the first air valve 4 from 87% to 107%; and (3) increasing the opening OP2 of the second air valve 5 from 64% to 82%. The multiple parameters were adjusted simultaneously. However, since the adjusting mode of the opening OP1 of the first air valve 4 >100%, it exceeded a set normal adjusting range. Therefore, the obtained optimal adjusting mode was that the air quantity FL of the fan 3 was adjusted from 43% to 58% and the opening OP2 of the second air valve 5 was adjusted from 64% to 82% simultaneously, such that the dissolved oxygen concentration DO1 of the first biochemical tank 1 and the dissolved oxygen concentration DO2 of the second biochemical tank 2 were increased simultaneously. DO1 and DO2 were maintained in a range of 2.3-2.7 mg/L after the adjustment.

### Comparative Example 1

The present comparative example provides a method for manually controlling dissolved oxygen of an aerobic wastewater treatment system specifically as follows: In the present comparative example, the aerobic wastewater treatment system used an activated sludge process to treat wastewater of an industrial park, a chemical oxygen demand range of influent was 100-500 mg/L, the aerobic wastewater treatment system comprised a first biochemical tank and a second biochemical tank, a group of variable frequency fans were used for daily oxygen supply of the first biochemical tank and the second biochemical tank, and the first air valve and the second air valve were respectively arranged before air led to the two biochemical reactors. An online influent quality and quantity data detector was used for monitoring influent quantity, chemical oxygen demand and an ammonia nitrogen concentration of the aerobic wastewater treatment system in real time, wherein an acquisition interval of data of the influent quantity was 30 minutes, and an acquisition interval of data of the chemical oxygen demand and the ammonia nitrogen concentration was 1 hour. A first online dissolved oxygen detector and a second online dissolved oxygen detector was respectively arranged in the first biochemical tank and the second biochemical tank, and an acquisition interval of data in real time was 1 minute. An acquisition interval of data of air quantity of the fan and openings of the two air valves was also 1 minute. The monitored various data were transmitted to a central control system in real time so as to facilitate observation and operation of workers.

The aerobic wastewater treatment system in the comparative example adjusted the dissolved oxygen of the first biochemical tank and the second biochemical tank by manpower in daily life. Workers conducted manual experience adjustment on the air valves and the air quantity of the fan in real time by observing a real-time dynamic change trend of the dissolved oxygen. When the air quantity of the fan was increased, both aeration quantities of a first biochemical tank and a second biochemical tank were increased, the dissolved oxygen concentrations were increased, and decreasing the air quantity of the fan in the same way. When the air valve opening of the first biochemical tank was controlled to be increased, the aeration quantity of the first biochemical tank was increased, and the dissolved oxygen concentration was increased. However, since the air quantity of the fan was unchanged, the aeration quantity of the second biochemical tank was reduced and the dissolved oxygen concentration was decreased. The change in the dissolved oxygen within 5 hours by the manual adjustment was tested.

The results showed that the method can maintain the dissolved oxygen concentrations DO1 and DO2 in the first and second biochemical tanks at 1.5-3.0 mg/L (FIG. 5). Although the manual adjusting method was more energy-saving than non-adjusting method, a fluctuation range of the dissolved oxygen in the first biochemical tank and the second biochemical tank was still larger, and the operation cost was high.

However, in example 1, after the system for accurately controlling dissolved oxygen of the present invention was connected, the dissolved oxygen was controlled to be 2.3-2.7 mg/L (FIG. 4) and the aim that the dissolved oxygen in the first biochemical tank and the second biochemical tank was maintained within a range of 0.2 mg/L of the set target value was achieved. In addition, the control range of the dissolved oxygen was obviously narrowed compared with that of the manual adjustment, the aims of more accuracy, energy saving and consumption reduction compared with the manual adjustment were achieved.

The present invention and the embodiments thereof are schematically described as above, and the description is not limiting; and what is shown in the examples is also only one of the embodiments of the present invention, and the actual embodiments are not limited thereto. Therefore, embodiments and examples, similar to the technical solution, designed without creative efforts by a person of ordinary skill in the art under inspiration without departing from the creative purposes of the present invention shall fall within the protection scope of present invention.

What is claimed is:

1. A method for accurately controlling dissolved oxygen of an aerobic wastewater treatment system, comprising the following steps:
S1, collecting data of influent quality, influent quantity, dissolved oxygen concentrations of a first biochemical tank and a second biochemical tank, a first air valve opening, a second air valve opening, and air quantity of a fan of an aerobic wastewater treatment system by using a data acquisition and control module, and transmitting the data to a server;
S2, constructing a machine learning matrix on the basis of the data of the influent quality, the influent quantity, the dissolved oxygen concentrations of the first biochemical tank and the second biochemical tank, the first air valve opening, the second air valve opening, and the air quantity of the fan of the aerobic wastewater treatment system;
S3, establishing a deep learning model among the influent quality, the influent quantity, the dissolved oxygen concentrations of the first biochemical tank and the second biochemical tank, the first air valve opening, the second air valve opening, and the air quantity of the fan of the aerobic wastewater treatment system; and training the deep learning model on the basis of the data matrix constructed in step S2;
S4, setting target values of the dissolved oxygen concentrations of the first biochemical tank and the second biochemical tank in the server; and
S5, when the dissolved oxygen concentrations of the first biochemical tank and the second biochemical tank deviate from the target values, calculating suggested adjustment values of the first air valve opening, the second air valve opening, and the air quantity of the fan on the basis of the model constructed in step S3, and maintaining the dissolved oxygen concentrations of the first biochemical tank and the second biochemical tank at the target values by adjusting the first air valve opening, the second air valve opening, and the air quantity of the fan.

2. The method for accurately controlling dissolved oxygen of an aerobic wastewater treatment system according to claim 1, wherein in step S1, the data of the influent quality comprises data of chemical oxygen demand concentration and ammonia nitrogen concentration of influent.

3. The method for accurately controlling dissolved oxygen of an aerobic wastewater treatment system according to claim 1, wherein in step S1, an acquisition time interval of the data of the influent quality, the influent quantity, the dissolved oxygen concentrations of the first biochemical tank and the second biochemical tank, the first air valve opening, the second air valve opening, and the air quantity of the fan of the aerobic wastewater treatment system is fixed value,
wherein, the acquisition time interval of the data of the influent quality and the influent quantity is less than or equal to 60 minutes, and the acquisition time interval of the data of the dissolved oxygen concentrations of the first biochemical tank and the second biochemical tank, the first air valve opening, the second air valve opening, and the air quantity of the fan is 20-60 seconds.

4. The method for accurately controlling dissolved oxygen of an aerobic wastewater treatment system according to claim 1, wherein in step S2, constructing the machine learning matrix is as follows:
$$[ \begin{matrix}{t}_{1} & OP {1}_{1} & OP {2}_{1} & {FL}_{1} & {COD}_{1} & {N}_{1} & int {FL}_{1} & DO {1}_{1} & DO {2}_{1} \\ {t}_{2} & OP {1}_{2} & OP {2}_{2} & {FL}_{2} & {COD}_{2} & {N}_{2} & int {FL}_{2} & DO {1}_{2} & DO {2}_{2} \\ ⋮ & ⋮ & ⋮ & ⋮ & ⋮ & ⋮ & ⋮ & ⋮ & ⋮ \\ {t}_{m} & OP {1}_{m} & OP {2}_{m} & {FL}_{m} & {COD}_{m} & {N}_{m} & int {FL}_{m} & DO {1}_{m} & DO {2}_{m}\end{matrix} ]$$
t₁-tₘ are the time of each record, OP1₁-OP1ₘ are the first air valve opening corresponding to each record, OP2₁-OP2ₘ are the second air valve opening corresponding to each record, FL₁-FLₘ are the air quantities of the fan corresponding to each record, COD₁-CODₘ are chemical oxygen demands of influent corresponding to each record, N₁-Nₘ are ammonia nitrogen concentrations of the influent corresponding to each record, intFL₁-intFLₘ are influent quantities corresponding to each record, DO1₁-DO1ₘ are the dissolved oxygen concentrations of the first biochemical tank corresponding to each record, and DO2₁-DO2ₘ are the dissolved oxygen concentrations of the second biochemical tank corresponding to each record.

5. The method for accurately controlling dissolved oxygen of an aerobic wastewater treatment system according to claim 1, wherein the deep learning model in step S3 is a long short-term memory model constructed based on a long short-term memory LSTM network.

6. The method for accurately controlling dissolved oxygen of an aerobic wastewater treatment system according to claim 5, wherein the long short-term memory model requires more than or equal to 6 days of data to train before use.

7. The method for accurately controlling dissolved oxygen of an aerobic wastewater treatment system according to claim 5, wherein in a use process of the long short-term memory model, training and learning are performed again every 30 minutes according to the updated data.

8. The method for accurately controlling dissolved oxygen of an aerobic wastewater treatment system according to claim 1, wherein in step S4, a target value range of the dissolved oxygen concentrations of the first biochemical tank and the second biochemical tank is 1-5 mg/L; and when a difference value of the measured dissolved oxygen concentration value and the target value is greater than 0.2 mg/L, the dissolved oxygen concentrations of the first biochemical tank and the second biochemical tank are considered to deviate from the target value.

9. The method for accurately controlling dissolved oxygen of an aerobic wastewater treatment system according to claim 1, wherein in step S5, when the air quantity of the fan needs to be adjusted to be greater, if the suggested adjustment values of the first air valve opening and/or the second air valve opening given by the server are within adjustable ranges, the first air valve opening or the second air valve opening is adjusted; and if the suggested adjustment values of the first air valve opening and the second air valve opening given by the server exceed the adjustable ranges, and the suggested adjustment value of the air quantity of the fan given by the server is within an adjustable range, the air quantity of the fan is adjusted;
when the air quantity of the fan needs to be adjusted to be lower, if the suggested adjustment value of the air quantity of the fan given by the server is within the adjustable range, the air quantity of the fan is adjusted; and if the suggested adjustment value of the air quantity of the fan given by the server exceeds the adjustable range, and the suggested adjustment values of the first air valve opening and/or the second air valve opening given by the server are within the adjustable ranges, the first air valve opening or the second air valve opening is adjusted;
when the air quantity of the fan and the openings of the first air valve and the second air valve given by the server are both suggested to be adjusted to be greater or lower by the server, a multi-parameter adjustment strategy is implemented, the three parameters are simultaneously determined whether the adjustable ranges are exceeded or not, and the parameters which are not exceeded are simultaneously adjusted; and
when the air quantity of the fan, and the suggested adjustment values of the first air valve opening and the second air valve opening provided by the server all exceed adjustable ranges, manual checking and confirmation are performed.

10. The method for accurately controlling dissolved oxygen of an aerobic wastewater treatment system according to claim 1, wherein adjustable ranges of the first air valve opening and the second air valve opening are 30-100%; and
an adjustable range of the air quantity of the fan is 20-90% of a maximum air quantity.

11. The method for accurately controlling dissolved oxygen of an aerobic wastewater treatment system according to claim 9, wherein
when the air quantity of the fan needs to be adjusted to be greater, if the suggested adjustment values of the first air valve opening and the second air valve opening given by the server are both within the adjustable ranges, the air valve that is suggested for a larger adjustment is adjusted.

12. The method for accurately controlling dissolved oxygen of an aerobic wastewater treatment system according to claim 11, wherein
when the air quantity of the fan needs to be adjusted to be greater, if the suggested adjustment values of the first air valve opening and the second air valve opening given by the server are both within the adjustable ranges, the first air valve opening is suggested to be adjusted to be greater by the server, and the second air valve opening is suggested to be adjusted to be lower, then the first air valve opening is adjusted.

13. The method for accurately controlling dissolved oxygen of an aerobic wastewater treatment system according to claim 11, wherein
when the air quantity of the fan needs to be adjusted to be greater, if the suggested adjustment values of the first air valve opening and the second air valve opening given by the server are both within the adjustable ranges, the first air valve opening is suggested to be adjusted to be lower by the server, and the second air valve opening is suggested to be adjusted to be greater, then the second air valve opening is adjusted.

14. The method for accurately controlling dissolved oxygen of an aerobic wastewater treatment system according to claim 9, wherein
when the air quantity of the fan needs to be adjusted to be lower, if the suggested adjustment value of the air quantity of the fan given by the server exceeds the adjustable range, and the suggested adjustment values of the first air valve opening and the second air valve given opening by the server are both within the adjustable ranges, the air valve that is suggested for a smaller adjustment is adjusted.

15. The method for accurately controlling dissolved oxygen of an aerobic wastewater treatment system according to claim 14, wherein
when the air quantity of the fan needs to be adjusted to be lower, if the suggested adjustment value of the air quantity of the fan given by the server exceeds the adjustable range, the suggested adjustment values of the first air valve opening and the second air valve opening given by the server are both within the adjustable ranges, the first air valve opening is suggested to be adjusted to be greater by the server, and the second air valve opening is suggested to be adjusted to be lower, then the second air valve opening is adjusted.

16. The method for accurately controlling dissolved oxygen of an aerobic wastewater treatment system according to claim 14, wherein
when the air quantity of the fan needs to be adjusted to be lower, if the suggested adjustment value of the air quantity of the fan given by the server exceeds the adjustable range, the suggested adjustment values of the first air valve opening and the second air valve opening given by the server are both within the adjustable ranges, the first air valve opening is suggested to be adjusted to be lower by the server, and the second air valve opening is suggested to be adjusted to be greater, then the first air valve opening is adjusted.
